(19) United States
(12) Patent Application Publication
(10) Pub. No.: US 2007/0027386 A1
(43) Pub. Date: Feb. 1, 2007

(54) Portable electronic device and a health management system arranged for monitoring a physiological condition of an individual

(75) Inventors: Olaf Such, Aachen (DE); Josef Lauter, Geilenkirchen (DE); Harald Reiter, Aachen (DE); Ralf Schmidt, Aachen (DE); Amdras Montvay, Stuttgart (DE); Jens Muehlsteff, Aachen (DE)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., Eindhoven (NL)

(21) Appl. No.: 10/564,388

(22) PCT Filed: Jul. 8, 2004
(86) PCT No.: PCT/IB04/51159

§ 371 Date: Jan. 12, 2006

(30) Foreign Application Priority Data
Jul. 16, 2003 (EP) 03077233.9

(51) Int. Cl. A61B 5/04

(52) U.S. Cl. 600/372; 600/509; 128/903; 600/393

(57) ABSTRACT

The invention relates to a portable electronic device, like a shaver, a toothbrush, a walkman, a telephony unit, etc., said device being arranged to measure a signal representative of a physiological condition of a user during a conventional usage of said device. In an embodiment of the electric shaver (25), it is provided with a first contact surface (26) comprising a plurality of shaving heads (26a, 26b, 26c). The shaving heads are manufactured from an electrically conducting material, usually a metal and are suited to provide a good electrical contact to the individual's skin during shaving, thus constituting a first electrode. The second contact surface (28′) is provided on the housing of the shaver, in particular on a grip portion (28) thereof, where a contact to a hand of the individual is enabled. The second contact surface (28′) comprises a second electrode (29). Additionally, the second contact surface (28′) can comprise a further sensor (29′) arranged to provide additional data on the physiological condition of the user. The signal measured from the electrodes is supplied to the input of the amplifier (30), which is preferably a differential amplifier. The signal from the differential amplifier (30) is then supplied to a band-pass filter (32), which is preferably set for the range of 0.02 Hz to 100 Hz. The limited amplified biosignal (33) is then forwarded to the analogue-to-digital converter (34). The digitized signal is then analyzed by the analysis means (35), the results of the analysis, comprising the deduced health-related parameter is being displayed on a display (36) of the electric shaver. Additionally the health-related parameter and/or the raw data are transmitted to a remotely arranged unit by a built-in transmission means (38). Preferably, the transmission means (38) comprises a wire-less transmitter.

The invention relates to a portable electronic device arranged to be brought into a contact with an individual's skin when being used by said individual, said device comprising a first contact surface and a second contact surface.

The invention still further relates to a health management system arranged to monitor a physiological condition of an individual, said system comprising:
sensing means arranged to detect a signal representative of said condition,
analysis means arranged to analyze said signal in order to derive a health-related parameter,
transmission means actuatable by said analysis means, said transmission means being arranged to forward said parameter to a remote health provider, said health provider being arranged to process said parameter in order to derive a health condition of said individual.

An embodiment of a device as set forth in the opening paragraph is known from WO 02/058307. The known device is a toothbrush arranged to provide a pulse oximetry measurement when being used by the individual. Pulse oximetry is an optical method using two-wavelength reflectance from fingertips of the individual to provide data related to a health condition of the individual. The principle of pulse oximetry is based on the red and infrared light absorption characteristics of oxygenated and deoxygenated haemoglobin. Oxygenated haemoglobin absorbs more infrared light and allows more red light to pass through. Deoxygenated (or reduced) haemoglobin absorbs more red light and allows more infrared light to pass through. Red light is in the 600-750 nm wavelength light band. Infrared light is in the 850-1000 nm wavelength light band. Pulse oximetry uses a light emitter with red and infrared light emitting diodes (LEDs) that shines through a reasonably translucent site with good blood flow. Typical adult/pediatric sites are the finger, toe, pinna (top) or lobe of the ear. Infant sites are the foot or palm of the hand and the big toe or thumb. For transmission measurement a photodetector is arranged opposite the emitter. The photodetector receives the light that passes through the tissue situated between the emitter and the photodetector. After the transmitted red (R) and infrared (IR) signals pass through the measuring site and are received at the photodetector, the R/IR ratio is calculated. The R/IR ratio can then be compared to a “look-up” table (made up of empirical formulas) that convert the ratio to an SpO₂ value. Most calibration curves used to fill the look-up table are derived from healthy subjects at various SpO₂ levels. When the R/IR ratio is established, a conclusion about the blood oxygenation level can be drawn. An application of this measurement principle to a class of handle-shaped devices for transtelephonic diagnosis is known per se in the art.

It is a disadvantage of the known device that the signal from only the fingertips of the individual is used to assess a health condition of the individual. In the measurement set-up using the fingertips, a reading with a relatively low reliability is achieved, as the pulse signal on red and infrared light is very sensitive to movement. Next to this, as the results of oximetry measurement relate to a blood oxygenation level, a direct conclusion about minor fluctuations in the health condition can hardly be drawn.

It is a purpose of the invention to provide an electronic device which is frequently used by the individual, where said device is adapted for measuring a signal representative to the health condition of the individual with improved signal quality and reliability of the measurement. It is a further object of the invention to provide an electronic device arranged to reliably measure minor fluctuations in the health condition of the individual.

The electronic device according to the invention is thus arranged so that the first contact surface comprises a first electrode and the second contact surface comprises a second electrode, said first electrode being electrically isolated from said second electrode; the device further comprising means for measuring an electrical signal from said first electrode and said second electrode during the usage of said device, said electrical signal being representative of a physiological condition of said individual.

The technical measure of the invention is based on the insight that a class of electrical appliances suited for personal care, personal entertainment or communication, provide a plurality of surfaces which are conceived to be brought into a contact with the individual's skin. Examples of suitable electrical appliances comprise a shaver, a walkman or any other like entertainment device, a telephony unit, etc. By making these surfaces electrically conducting, the electrical signal representative to a physiological condition of the individual can be measured during a usage of such a device thus combining a primary usage of such a device with a secondary usage, namely a measuring and/or monitoring of a physiological condition of the individual. In particular, a measurement of an electrocardiogram can be enabled by means of such a device.

In an embodiment of the device according to the invention, said device comprises analysis means arranged to perform an analysis of said electrical signal in order to derive a health-related parameter. In case the measured signal is related to the electrocardiogram, it is found to be advantageous to provide the device with analysis means arranged to analyze the acquired ECG spectrum. For example, the analysis means can be arranged to perform a pulse rate analysis, by means of calculating a repetition rate of a characteristic peak in the ECG spectrum. Preferably, a R-peak is used for that purpose. Alternatively, the analysis means can be arranged to perform a trend analysis of the ECG spectrum, for example by means of comparing an actual measurement to a measurement stored in a memory of the device. In order to enable this function the electronic device is provided with a memory chip accessible by a micro-controller of the device.

In a further embodiment of the device according to the invention, said device further comprises a user interface connectable to said analysis means for representing said health-related parameter to the individual. It is advantageous to provide a feed-back to the user about the measurement which is performed by the device. It is particularly advantageous to provide this feed-back to low risk patients as a measure of a daily prophylactic check-up.

In a still further embodiment of the device according to the invention, said device comprises transmission means arranged to forward said health-related parameter to a remotely arranged unit. Preferably, said transmission is enabled to another device, which is, for example, located at the individual's premises for purposes of durable storage and later reporting of measurement data. For example, the electronic device of the invention can be arranged to transmit the health-related parameter, for example a heart rate to a dedicated hardware, like a computer, a mobile phone, a data connection port, a personal digital assistant or any other suitable hardware. Preferably, the transmission of the health-related parameter is carried-out by means of a wireless technology. Examples of for this purpose suitable wireless technology are Bluetooth and DECT. Wireless technologies are known per se for a person skilled in the art and will not be elaborated further.

In a health management system according to the invention the sensing means comprise a portable electronic device arranged to be brought into a contact with an individual's skin when being used by said individual, said device comprising a first contact surface and a second contact surface, wherein the first contact surface comprises a first electrode and the second contact surface comprises a second electrode, said first electrode being electrically isolated from said second electrode; the device further comprising means for measuring an electrical signal from said first electrode and said second electrode during the usage of said device, said electrical signal being representative of a physiological condition of said individual.

In the health management system according to the invention use is made of an electronic device which is conceived to be frequently used by the individual for purposes of daily grooming, entertainment or communication. By providing such a device with suitable sensing means a monitoring of the health condition of said individual is enabled in an easy, cost-effective yet reliable way. In case the sensing means are integrated into a electronic shaver or a toothbrush, the health condition of the individual is subjected to structured prophylactic check-ups at approximately the same hour of the day and at similar environment. The individual using such an electronic device does not have to take additional measures to perform a daily health check-up, which is of particular advantage for low risk patients. Systems arranged for monitoring a health condition of the individual are known in the art. In the health management system according to the invention use is made of per se known hardware for analyzing the measured signal in order to deduce the health-related parameter as well as of known transmission means arranged for transmitting said parameter to a remotely arranged health care provider.

In an embodiment of the health management system according to the invention, the transmission means is arranged to transmit the health-related parameter by means of a wireless signal to a base unit arranged to enable a connection to the medical care provider by means of a communication network. For patients which are under a polyclinic observation, it is advantageous to make available the measurement data to a medical specialist, located at the remote medical care center. The base unit according to the invention receives the health-related parameter from the transmission means, it being, for example, a pulse rate of the individual. Also, a transmission of full measurement data can be enabled for purposes of an inspection by the medical specialist. It is even possible that by doing this, the patient under observation does not have to visit the medical care provider for a regular check-up, which increases the quality of life of the patient and decreases the workload of the medical care provider.

These and other aspects of the invention will be discussed in further detail with reference to Figures.

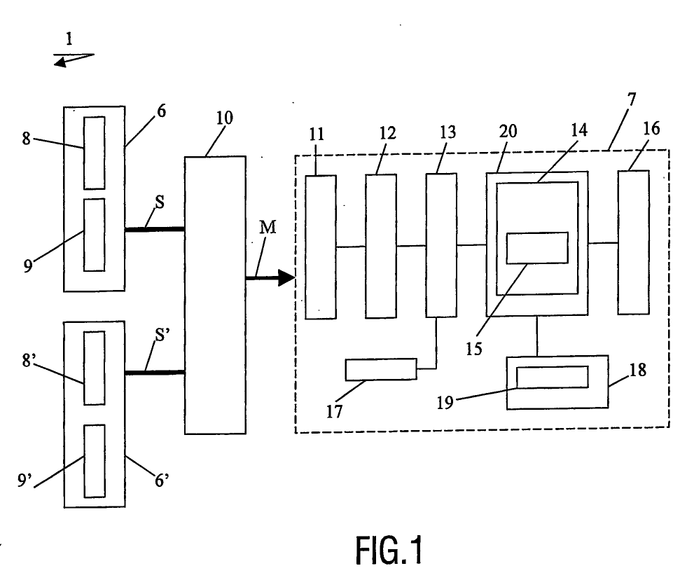

FIG. 1 presents a schematic view of an embodiment of the electronic device according to the invention.

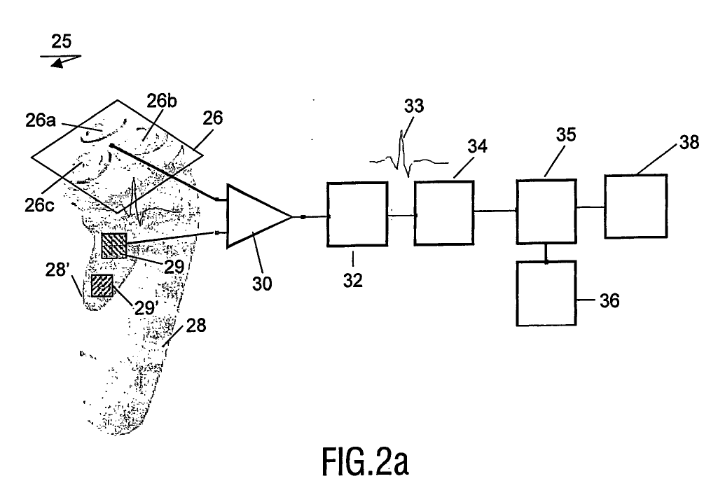

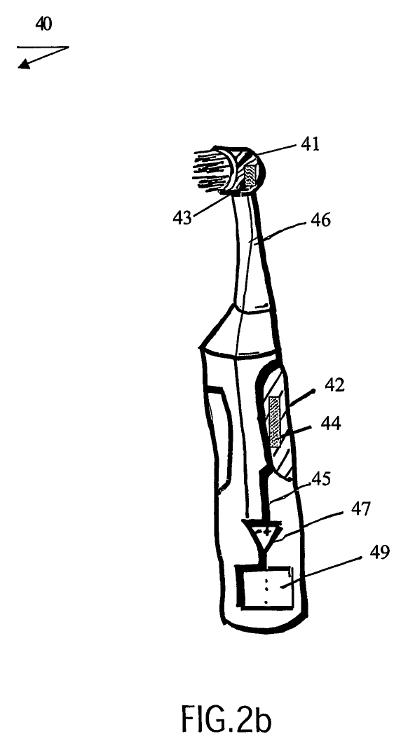

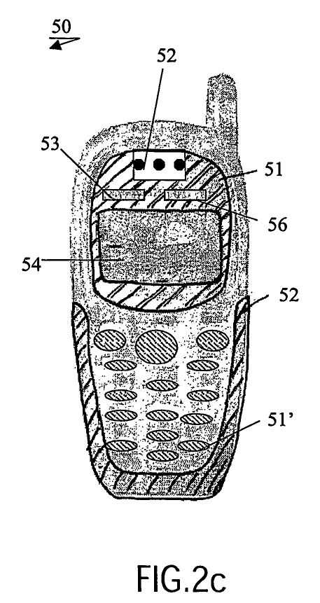

FIG. 2a presents a schematic view of an embodiment of an electric shaver arranged for measuring the electrical signal representative to the physiological condition of the individual.

FIG. 2b presents a schematic view of an embodiment of an electric toothbrush arranged for measuring the electrical signal representative to the physiological condition of the individual.

FIG. 2c presents a schematic view of an embodiment of a mobile phone arranged for measuring the electrical signal representative to the physiological condition of the individual.

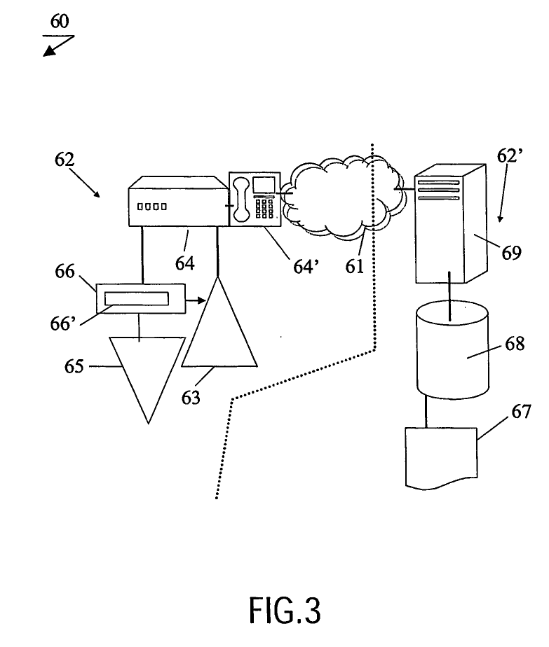

FIG. 3 presents a schematic view of an embodiment of a health management system according to the invention.

FIG. 1 presents a schematic view of an embodiment of the electronic device 1 according to the invention.

The electronic device 1 comprises the first contact surface 6 arranged to enable a first contact area with the individual's skin. The electronic device 1 comprises further the second contact surface 6′ arranged to enable a second contact area with the individual's skin. The first contact area 6 and the second contact area 6′ are provided with the first electrode 8 and a second electrode 8′, respectively. The electrodes 8, 8′ are arranged to provide an electrical contact to the individual's skin in order to perform a measurement of an electrical signal related to the physiological condition of the individual. The signal S, S′ respectively from the electrodes is supplied to the means for measuring the electrical signal 10. Additionally, the device 1 can comprise a sensor arranged to monitor a signal not directly related with a targeted physiological condition, for example an oximetry sensor, 9,9′, respectively. The means for measuring the electrical signal 10 are arranged to perform a measurement of the electrical signal directly related to the health condition of the individual by performing a necessary power supply to the sensors 8,8′,9,9′. The means for measuring the electrical signal 10 are further arranged to provide a corresponding signal M to the front-end electronics 7 of the device 1. The front-end electronics 7 is arranged to analyze said signal in order to derive a health-related parameter. For that purpose the front-end electronics 7 comprise a preamplifier and analogue processing circuit 11, an ADC unit 12, a μ-processor 13, detection means 20 and transmission means 16. The analysis means 20 comprise a sensor signal interpretation unit 14 provided with parameter extraction means 15. The device 1 operates as follows: when the device is being used by the individual and is powered for that purpose, the means for measuring the electrical signal 10 provide the necessary power supply to the sensors 8,8′,9,9′. When the contact surfaces 6,6′ are making put in contact with the individual's skin, the electrodes 8,8′,9,9′ provide a corresponding input signal to the means for measuring the electrical signal 10. The measured signal is made available to the front-end electronics 7. The front-end provides means for receiving the signals from the sensing means, performs suited analogue processing by means of the analogue processing circuit 11. The processed raw data is converted into a digital format by means of the ADC 12 and is forwarded by a μ-processor 13 to the analysis means 20, where the condition of the user is being analyzed. For example, for cardiac applications the analysis means 20 can comprise a per-se known QRS-detector to determine R-R peak intervals in heart cycles. The analysis means 20 comprise a sensor signal interpretation unit 14 arranged to derive a health-related parameter (15). For example, for cardiac applications said feature can be a frequency of the signal. It is also possible that more than one health-related parameter is assigned per monitored physiological condition. In this case the parameter can be ranked up according to the severity of, for example the detected abnormality in the physiological condition. For example, for cardiac applications, a minor change in the cardiac cycle can be recognized as a warning of the lowest category, whereas an occurrence of arrythmia or fibrillation can be ranked higher. Preferably, the value of the health-related parameter corresponding to a normal condition of the individual is stored in a look-up table (not shown) of the memory unit 17. Additionally, the system can be arranged as a self-learning system, where a threshold value for the health-related parameter is being adjusted and stored in the look-up table in cases a pre-stored value does not correspond to an abnormal condition for a particular user.

The analysis means 20 are further arranged to provide the health-related parameter to the user-interface 18 for the convenience of the user. The user-interface 18 is preferably a part of a display, which is standard for most portable electronic devices. The determined actual health-related parameter, for example a pulse rate is then displayed in a suitable window 19. In case the analysis means 20 detects the abnormal condition, a signal is sent to the user interface 19 to generate an alarm. A suitable way of alarming is an audio alarm or a light-alarm. The transmission means 16, can be arranged to transmit the alarm to the base unit (not shown), for example by means of a RF-link. From the respective station the emergency center is informed. The alarm center takes over the management of the emergency and informs the respective communal or medical sites about the emergency, the location, patient data and a probable diagnosis. Additionally, in case the device 1 is an electric shaver or an electric toothbrush, the alarm and/or the reading of the health-related parameter can be transmitted to a suitably arranged bathroom mirror, for example by means of Bluetooth or other short range wireless communication.

FIG. 2a presents a schematic view of an embodiment of an electric shaver arranged for measuring the electrical signal representative to the physiological condition of the individual. The electric shaver 25 is provided with a first contact surface 26 comprising a plurality of shaving heads 26a, 26b, 26c. The shaving heads are manufactured from an electrically conducting material, usually a metal and are suited to provide a good electrical contact to the individual's skin during shaving. The second contact surface 28′ is provided on the housing of the shaver, in particular on a grip portion 28 thereof, where a contact to a hand of the individual is enabled. The second contact surface 28′ comprises a second electrode 29. Preferably, the second electrode is manufactured from a conductive rubber and is shaped to accommodate a thumb of the individual. This minimizes movement artifact during shaving and improves the measured signal. Additionally, the second contact surface 28′ can comprise an oximetry sensor 29′ arranged to provide additional data on the physiological condition of the user. The signal measured from the electrodes is then supplied to the input of the amplifier 30, which is preferably a differential amplifier. The signal from the differential amplifier 30 is then supplied to a band-pass filter 32, which is preferably set for the range of 0.02 Hz to 100 Hz. The limited amplified biosignal 33 is then forwarded to the analogue-to-digital converter 34. The digitized signal is then analyzed by the analysis means 35, the results of the analysis, comprising the deduced health-related parameter is being displayed on a display 36 of the electric shaver. Additionally the health-related parameter and/or the raw data are transmitted to a remotely arranged unit by a built-in transmission means 38. Preferably, the transmission means 38 comprises a wire-less transmitter.

Additionally, it is possible to provide the electrical shaver with a plurality of operation modes, where the first operation mode corresponds to a set-up discussed above. The second operation mode corresponds to a state where the shaving heads are not moved, and comprise the first electrode and the second electrodes. In this case an additional wiring to the shaver heads is provided which is activated upon a selection of the second mode of operation. In this mode the shaver can measure an ECG when positioned on the chest of the individual. The electrode 29 in this case serves as a reference electrode to improve the signal quality. The second mode is particular advantageous as signal interferences occurring during a normal operational mode are avoided.

FIG. 2b presents a schematic view of an embodiment of an electric toothbrush 40 arranged for measuring the electrical signal representative to the physiological condition of the individual. The head 41 of the toothbrush 40 is preferably manufactured of a conductive plastic or a metal or a suitable coating of conductive silicone rubber. The fibers of the toothbrush may still be fabricated of a non-conductive material ensuring a good care for the gums, because the head of the toothbrush is located in a wet environment during a procedure of a toothbrushing, therefore a good signal conductance to the head of the toothbrush from the body of the individual is enabled. The head of the toothbrush 41 thus constitutes the first electrode. A miniaturized electrode 43 can be provided on the surface of the head. Alternatively, it is possible that the whole surface area of the brush head constitutes the electrode to ensure a good signal conductance. The handle 48 of the electric toothbrush 40 is provided with a grip portion 42, which is conceived to enable a contact with the individual's hand. The grip portion 42 comprises the second contact surface with the second electrode 44 thus enabling the electrical contact with the individual's skin. The wiring 46, 45 from the first electrode 41 and the second electrode 42, respectively, provide the measured electrical signal to the front-end electronics 47. An embodiment of a suitable front-end electronics is discussed with reference to FIG. 1. The front-end electronics carries-out a suitable signal analysis and supplies the health-related parameter to the display unit 49.

FIG. 2c presents a schematic view of an embodiment of a mobile telephony unit 50 arranged for measuring the electrical signal representative to the physiological condition of the individual. The mobile telephony unit 50 comprises a first contact surface 51 arranged on a housing of the mobile telephone unit 50 in the area in the direct vicinity of an earpiece 52. The first contact surface is manufactured from an electrically conducting material, preferably a conducting plastic. The first contact surface comprises the first electrode 53, arranged to measure an electrical signal from the individual's skin. Additionally, the first contact surface 51 may comprise a further sensor 56 to measure an additional signal related to the physiological condition of the individual. An example of the suitable further sensor is an oximeter. As is shown in the introductory part of the application, the oximetry measurements are particularly suited to be carried out on the ears.

Alternatively, the first contact surface can be on the keypad 51′, as it is known that during a telephone conversation, telephony unit makes a good contact with the individual's cheek. It is common to manufacture the keypad with a metal coating, to ensure a durable usage of the keys. By providing a key or a plurality of keys with a suitable wiring, the electrical signal representative of the physiological condition of the individual can be measured. This embodiment is cheap to produce.

The housing of the telephony unit 50 is provided with a grip portion 52, where the individual is supposed to hold the telephony unit during its usage, thus constituting the second contact surface. Preferably, the area 52 is manufactured from a conductive material thus acting as the second electrode. It is also possible to make the second electrode smaller is size, so that it takes only a part of the second contact surface 52. By providing a suitable wiring, the electrical signal representative of the physiological condition of the individual can be measured. The signal is then processed by the front-end electronics (not shown) and the deduced health-related parameter is shown to the individual on the display 54.

FIG. 3 presents a schematic view of an embodiment of a health management system according to the invention. The health management system 60 comprises a user-site 62 connectable to a remotely arranged medical care provider 62′ by means of a telecommunication line 61. The user-site comprises a home station 64 arranged with a telephone module 64′. An event that a user is in a need of a medical assistance, is notified by a trigger means 63 arranged to forward the trigger call by means of the telephone module 64′ to the remotely arranged medical care provider 62′. The trigger means 63 is actuated by the analysis means 66 arranged to analyze a signal representative of a medical condition of the individual, said signal being provided by a monitoring system 65 comprising the electronic device, as is discussed with reference to FIG. 1. The signal from the electronic device (not shown) is analyzed by the analysis means 66 and a health-related parameter 66′ is deduced. The trigger means 63 is actuated in case the health-related parameter falls outside a normal range. The trigger means 63 is further arranged to provide an identification of the user to the remote medical care provider 62′. An example of a suitable identification is a name. The telephony unit 64′ forwards the trigger call together with the user identification to the remotely arranged medical care provider 62′. The remotely arranged medical care provider 62′ uses a pre-stored information from a database 68 for uploading necessary background patient data 67. Preferably, the patient data comprise a history of a patient case, diagnosis and other suitable medically relevant data.

1. A portable electronic device (1) arranged to be brought into a contact with an individual's skin when being used by said individual, said device comprising a first contact surface (6) and a second contact surface (6′), wherein the first contact surface comprises a first electrode (8) and the second contact surface comprises a second electrode (8′), said first electrode being electrically isolated from said second electrode; the device further comprising means for measuring an electrical signal (10) from said first electrode and said second electrode during the usage of said device, said electrical signal being representative of a physiological condition of said individual.

2. A device according to claim 1, wherein said device further comprises analysis means (20) arranged to perform an analysis of said electrical signal (M) in order to derive a health-related parameter (15).

3. A device according to claim 2, wherein said device further comprises a user interface (18) connectable to said analysis means (20), said user interface being arranged to present said health-related parameter to the individual.

4. A device according to claim 3, wherein said device further comprises transmission means (16) arranged to forward said health-related parameter to a remotely arranged unit.

5. A device according to claim 1, wherein said device is arranged to measure an electrical signal generated by cardiac activity.

6. A device according to claim 5, wherein said device is an electric shaver (25), the first contact surface (26) comprising a front surface of a shaving head (26a, 26b, 26c), the second contact surface (28′) comprising a grip portion (28) of the shaver.

7. A device according to claim 5, whereon said device is an electric shaver (25) comprising a plurality of shaving heads (26a, 26b, 26c), the first contact surface comprising a first electrode (26a), the second contact surface comprising a second electrode (26b), the electrical shaver further comprising a grip portion (28), said portion being arranged to comprise a further electrode (29) conceived to provide a reference signal.

8. A device according to claim 5, wherein said device is an electric toothbrush (40), the first contact surface comprising a brush head (41), the second contact surface comprising a grip portion (42) of the toothbrush.

9. A device according to claim 5, wherein said device is a telephone handset (50), the first contact surface comprising a housing area (51) of the telephone handset, said area being arranged in a direct vicinity of an earpiece (53), the second contact surface comprising a grip portion (52) of the telephone handset (50).

10. A device according to claim 9, wherein said telephone handset is a mobile telephone handset (50), the first contact surface comprising a keypad (51′), the second contact surface comprising a grip portion (52) of the mobile telephone handset.

11. A device according to claim 5, wherein said device comprises an earphone and a body unit, the first contact surface being arranged on the earphone, the second contact surface being arranged on the body unit.

12. A health management system arranged to monitor a physiological condition of an individual, said system comprising
sensing means (65) arranged to detect a signal representative of said condition,
analysis means (66) arranged to analyze said signal in order to derive a health-related parameter (66′),
transmission means (64′) actuatable by said analysis means, said transmission means being arranged to forward said parameter to a remotely arranged medical care provider (62′), said provider being arranged to process said parameter in order to derive a health condition of said individual, wherein
said sensing means (65) comprise a portable electronic device arranged to be brought into a contact with an individual's skin when being used by said individual, said device comprising a first contact surface and a second contact surface, wherein the first contact surface comprises a first electrode and the second contact surface comprises a second electrode, said first electrode being electrically isolated from said second electrode; the device further comprising means for measuring an electrical signal from said first electrode and said second electrode during the usage of said device, said electrical signal being representative of a physiological condition of said individual.

13. A health management system according to claim 12, wherein the transmission means is arranged for transmitting said parameter by means of a wireless signal to a base unit arranged to enable a connection to the medical care provider by means of a communication network.

14. A health management system according to claim 12, wherein the device further comprises a user interface actuatable by the analysis means, said user interface being arranged to present said parameter to the individual.
